(19) United States
(12) Patent Application Publication
(10) Pub. No.: US 2017/0151192 A1
(43) Pub. Date: Jun. 1, 2017

(54) METHODS, COMPOSITIONS AND DEVICES FOR TREATMENT OF MOTOR AND DEPRESSION SYMPTOMS ASSOCIATED WITH PARKINSON'S DISEASE

(71) Applicant: N TO B LTD., Yokneam Ilit (IL)

(72) Inventors: Moussa B.H. YOUDIM, Nesher (IL); Orly WEINREB, Haifa (IL); Tamar AMIT, Haifa (IL)

(21) Appl. No.: 15/430,305

(22) Filed: Feb. 10, 2017

Related U.S. Application Data
(63) Continuation of application No. 14/774,181, filed on Sep. 10, 2015, now abandoned.
(60) Provisional application No. 61/779,854, filed on Mar. 13, 2013.

(51) Int. Cl. A61K 31/135; A61M 15/00; A61M 15/08; A61K 9/00; A61K 47/26

(52) U.S. Cl. CPC A61K 31/135; A61K 9/0043; A61K 9/0075; A61K 47/26; A61K 9/0078; A61M 15/08; A61M 15/009

(57) ABSTRACT

Disclosed is a method of treating Parkinson's disease and/or depression, and motor symptoms, non motor symptoms and depression symptoms associated with Parkinson's disease in a subject in need thereof, the method including administering to said subject a pharmaceutical composition comprising rasagiline or a pharmaceutically acceptable salt thereof, wherein the administering is effected by intranasal administration, and wherein a daily dose of the rasagiline or pharmaceutically acceptable salt thereof in the pharmaceutical composition is sufficient to inhibit monoamine oxidase (MAO)-A and MAO-B in a brain but insufficient to inhibit systemic MAO-A and therefore does not potentiate sympathetic cardiovascular activity associated with tyramine rich food consuming. Also, disclosed is a pharmaceutical composition including rasagiline or a pharmaceutically acceptable salt thereof and a pharmaceutically acceptable carrier, and a device configured for intranasal administration of a pharmaceutical composition including rasagiline or a pharmaceutically acceptable salt thereof to a subject.

### FIELD AND BACKGROUND OF THE INVENTION

The present invention, in some embodiments thereof, relates to therapy and, more particularly, but not exclusively, to compositions, methods and devices useful for the treatment of depression and/or motor and depression symptoms of Parkinson's disease.

Monoamine oxidase, also known as monoamine oxygen oxidoreductase, is abbreviated as MAO and has the EC NUMBER EC 1.4.3.4. This enzyme is known as oxidizing primary aliphatic and aromatic amines and some secondary and tertiary amines. The reaction catalyzed by MAO can be represented by the following generalized equation:

RCH₂NR′R″+O₂+H₂O→RCHO+NR′R″+H₂O₂

Two isoenzymes of monoamine oxidase are present in most mammalian tissues. These isoenzymes, denoted in the art as MAO-A and MAO-B, were originally distinguished by their sensitivities to inhibition by the acetylenic inhibitors clorgyline and deprenyl and by their substrate specificities.

Typically MAO-A catalyzes the oxidative deamination of 5-hydroxytryptamine (5-HT), whereas MAO-B is active toward benzylamine and 2-phenylethylamine (PEA), yet, these substrate specificities are not absolute, and several studies have been conducted in the past years in this respect. See, for example, O'Carroll et al., in Biochemical Pharmacology, Vol. 38, No. 6, pp. 910-905, 1989; and Green et al., in Br. J. Pahrmac., 1977, 60, 343-349.

In most species, tyramine and dopamine, as well as others, are substrates for both enzymes in the brain, and the role of MAO enzymes is recognized as regulating the metabolism of catecholamine neurotransmitters (e.g., dopamine and noradrenaline) and serotonin. A list of exemplary substrates of MAO-A and/or MAO-B is presented, for example, in Tipton et al., “Monoamine oxidase: functions in the central nervous system”, In Encyclopedia of Neuroscience, Adelman, G; B. Smith, B, Eds:. Elsevier Science BV, Amsterdam, 3rd Edition, 2004.].

MAO-A and/or MAO-B in peripheral tissues such as the intestine, liver, lung, and placenta appear to play a protective role in the body by oxidizing vasoactive amines from blood or preventing their entry into the circulation. In the central and peripheral nervous system, intraneuronal MAO-A and MAO-B have been suggested to protect neurons from exogenous amines and/or to regulate levels of neurotransmitter amines synthesized within a neuron.

As such, non selective MAO and selective MAO-A inhibitors were primarily considered as anti-depressants. Along the researches conducted, it was uncovered that more than 80% inhibition of MAO-A is required for increasing the concentration of 5-HT and noradrenaline in the brain and thus to exhibit antidepressant effects [See, for example, Tipton et al., 2004, supra].

However, the use of MAO-A selective or nonselective irreversible MAO inhibitors has been limited because individuals taking these drugs become susceptible to amines, such as tyramine, in the diet. These amines are normally degraded by MAO in peripheral tissues. When MAO is inhibited, ingested tyramine can enter the blood, from which it is taken up by adrenergic nerve terminals releasing stored noradrenaline and resulting in a hypertensive response (hypertensive crises). On the basis of the high concentrations of tyramine in some cheeses, this hypertensive response to dietary amines is widely known as the “cheese reaction” or “cheese effect”. Tyramine is found in high concentration also in other food nutrients.

Longer-term administration of such inhibitors can lead to hypotension, possibly as a result of the accumulation of false transmitters in the nerves.

Thus, due to side effects of the MAO-A inhibitory drugs, e.g., hypertension (“cheese effect”), these drugs are often replaced by other antidepressants [Youdim et al., Journal of neural transmission, 1987, 25, 27-33; Youdim, Journal of neural transmission, 1980, 16, 157-161].

Most studies have shown that selective MAO-B inhibitors such as (−)-deprenyl (Selegiline) exhibit no effect in patients suffering from endogenous depression, and further have a very low interaction with tyramine when given either orally or intravenously.

On the other hand, MAO inhibitors were found to be highly potent protectors against MPTP (1-methyl-4-phenyl-1,2,3,6-tetrahydropyridine) neurotoxicity, which gives rise to a condition resembling idiopathic Parkinson's disease. Inhibitors of MAO are therefore considered to prolong the actions of dopamine in Parkinson's disease.

Parkinson's disease (PD) is a slowly progressive neurodegenerative disorder. It is characterized by motor symptoms, such as bradykinesia, rigidity, tremor at rest and postural instability, which are associated with degeneration of the nigrostriatal dopaminergic projection originating in the substantia nigra (SN). In addition to these motor symptoms, non-motor symptoms, such as olfactory dysfunction, can be observed even prior to the manifestation of motor symptoms in PD patients.

Depression is a common psychiatric comorbidity in PD, affecting more than 60% of the patients. Pharmacotherapy for depression in PD entails special concerns related to different side effects of the various anti-depressants and great potential for drug-drug interaction. Selective serotonin reuptake inhibitors (SSRIs) are typically prescribed, however with often insufficient results.

Selegiline (also referred to in the art as Anipryl, (−)-deprenyl, L-deprenyl, Eldepryl, Emsam, Zelapar) and Rasagiline (N-propargyl-1-(R)-aminoindan; Marketed as Azilect®)) are dose-dependent selective irreversible inhibitors of MAO-B. As such they have found therapeutic applications for the treatment of Parkinson's disease (PD) and in the treatment of Major Depressive Disorder (MDD). Both drugs are orally-administered drugs, and are subject to extensive first pass hepatic metabolism, resulting in poor and highly variable oral bioavailability (35% for Rasagiline and 4-10% for Selegiline).

Although Rasagiline and Selegiline are preferential MAO-B inhibitors, their selectivity is dose-dependent; While Selegiline is approved for the treatment of depression, the dose is 3- to 6-fold higher than that for the treatment of PD, causing loss of MAO-B selectivity and requiring precautions to prevent hypertensive crises due to “cheese reaction”.

Rasagiline is a new MAO-B inhibitor, introduced in 2006, that has 3- to 16-fold greater potency than Selegiline. Similarly to Selegiline, Rasagiline is generally devoid of potential to cause hypertensive crises, the “cheese reaction”, unless administered at high concentrations that are sufficient to inhibit MAO-A [Youdim and Bakhle, 2006, Br J Pharmacol., 2006, 147 Suppl 1, S287-296].

Animal studies in rats showed that Rasagiline does not potentiate pressor responses to oral tyramine by single oral doses up to 5 mg/kg, or following 21 days of chronic treatment at oral doses up to 2 mg/kg daily. At 10 mg/kg oral dose of rasagiline, both MAO-A and MAO-B in rats are inhibited, and the irreversible inhibition of MAO-A induces the “cheese reaction” [Finberg et al., Selective irreversible propargyl derivative inhibitors of monoamine oxidase (MAO) without the cheese effect. In: Monoamine oxidase inhibitors—the state of the art. Youdim, M. B. H.; Peykel, E. S., eds. Chichester: Wiley; 1981a, 31-41; Finberg et al., Br J Pharmacol., 1981, 73, 65-74; Finberg and Youdim, Journal of Neural Transmission, 1988, Supplementum 26, 11-16; and Youdim, M. B. H., Expert Review of Neurotherapeutics, 2003, 3, 737-749].

The recommended dose of rasagiline for humans is 1 mg once daily when used alone (monotherapy), and 0.5-1 mg once daily when combined with 1-DOPA. Patients with mild liver disease should not use more than 0.5 mg daily.

Further, patients administered with Rasagiline are typically cautioned to avoid tyramine rich food, as well as administration of anti-depressants of the selective serotonin uptake inhibitors family, serotonin-norepinephrine uptake inhibitors family and other MAO inhibitors.Rasagiline is primarily metabolized by hepatic cytochrome P-450 to form its major metabolite, 1-(R)-aminoindan, a non-amphetamine, weak reversible MAO-B inhibitor compound. Recent studies indicated the potential neuroprotective effect of 1-(R)-aminoindan, suggesting that it may contribute to the overall neuroprotective and antiapoptotic effects of its parent compound, rasagiline. In contrast, one of the Selegiline principal metabolites is 1-methamphetamine which can be converted to 1-amphetamine. See, for example, Bar Am et al., Journal of Neurochemistry, 2010, Vol. 11, pp. 1131-1137; Weinreb et al, Antioxidants & Redox Signaling, Volume 14, Number 5, 2011, page 767.

There are several reports on administration of Rasagiline and/or Selegiline via routes other than the oral. These include, for example, Kalaria et al. [International Journal of Pharmaceutics 438 (2012) 202-208], which describe gel patches for transdermal delivery of Rasagiline and Selegiline; and Ravi et al, 2013 Drug Delivery: Pages 1-8 Nov. 29, 2013. which describe Intranasal thermosensitive gel for rasagiline mesylate (RM) delivery, which is reported to exhibit improved bioavailability over oral solutions.

Intranasal administration is a noninvasive means for targeting the brain bypassing the blood-brain barrier (BBB), minimizing systemic absorption, and limiting potential peripheral side effects [Vyas et al., Current drug delivery, 2005, 2, 165-175; Illum, 2004, The Journal of pharmacy and pharmacology, 2004, 56, 3-17].

Additional background art includes deMarcaida et al. Mov Disord, 2006, 21, 1716-1721; Goren et al., J Clin Pharmacol, 2010, 50, 1420-1428; Tipton et al., Biochemical Pharmacology, 1982, 31, 1251-1255; Ravaris et al., Arch Gen Psychiatry, Vol. 33, March 1976; U.S. Patent Application having Publication No. 2010/0189791; and U.S. Pat. Nos. 5,453,446 and 5,668,181.

### SUMMARY OF THE INVENTION

While MAO-B inhibitors such as Rasagiline are useful in the treatment of Parkinson's disease, particularly in alleviating motor symptoms associated with Parkinson's disease, a need still remains to treat depression symptoms associated with this disease. Such a need becomes even more pronounced due to adverse drug-drug interactions between Rasagiline and commonly used anti-depressants such as SSRIs, and while considering the depression symptoms associated with a majority of Parkinsonian patients.

While MAO-B inhibitors such as, for example, Rasagiline and Selegiline, may act also as anti-depressants at higher doses, due to MAO-A inhibition, administration of these drugs at doses effective in treating MAO-A inhibition is limited by the “cheese reaction” (hypertensive crisis) associated with such high doses.

Currently, Rasagiline and Selegiline are prescribed for the treatment of Parkinson at doses which are selective to MAO-B inhibition and are therefore ineffective in treating depression symptoms in Parkinsonian patients.

The present inventors have now designed and successfully practiced a novel methodology, in which a MAO-B inhibitor such as Rasagiline is administered into the brain at a range of doses, which are capable of affecting depressive illness (depression). According to this methodology, administering a MAO-B inhibitor such as Rasagiline to the brain of a subject results in alleviating or treating both motor and depression symptoms associated with Parkinson's disease. This methodology, for example, is effected while utilizing Rasagiline or any other MAO-B inhibitor, at doses sufficient to inhibit MAO-A in the brain, possibly without potentiation of sympathetic cardiovascular activity (e.g., “cheese reaction”). Such a methodology allows using drugs such as Rasagiline for exhibiting anti-depressant activity (e.g., due to MAO-A inhibition), and particularly, for exhibiting a dual effect of MAO-A and MAO-B inhibition, thereby improving the effect of drug in patients suffering from Parkinson's disease by alleviating both motor and depression symptoms associated with the disease.

The present inventors have demonstrated that intranasal administration of Rasagiline at a range of doses, results in both MAO-A and MAO-B inhibition in the brain, whereby similar doses, when administered intraperitonially or orally, are inefficient in inhibiting MAO-A in the brain. The present inventors have further demonstrated that intranasal administration of Rasagiline at a range of doses, results in efficient inhibition of MAO-A and MAO-B in the brain, yet in inefficient inhibition of MAO-A in the periphery (e.g., liver and small intestine).

According to an aspect of some embodiments of the present invention there is provided a use of Rasagiline or of a pharmaceutically acceptable salt thereof in the manufacture of a pharmaceutical composition for treating motor symptoms and depression symptoms associated with Parkinson Disease in a subject in need thereof, wherein the composition is formulated for intranasal administration of Rasagiline or a pharmaceutically acceptable salt thereof.

According to an aspect of some embodiments of the present invention there is provided a method of treating motor symptoms and depression symptoms associated with Parkinson's Disease in a subject in need thereof, the method comprising administering to the subject a pharmaceutical composition comprising Rasagiline or a pharmaceutically acceptable salt thereof, wherein the administering is effected by intranasal administration.

According to some embodiments of the present invention, the composition is formulated for intranasal administration such that an amount of rasagiline which is sufficient to inhibit MAO-A in the brain is administered.

According to some embodiments of the present invention, the composition is formulated for intranasal administration such that an amount of rasagiline or a pharmaceutically acceptable salt thereof is lower than an amount equivalent to 10 mg/kg per day in rats.

According to some embodiments of the present invention, the pharmaceutical composition further comprises a pharmaceutically acceptable carrier, the carrier being a solid carrier or a liquid carrier.

According to an aspect of some embodiments of the present invention there is provided a use of Rasagiline or of a pharmaceutically acceptable salt thereof in the manufacture of a pharmaceutical composition for treating Parkinson's disease and/or depression associated with Parkinson's disease in a subject in need thereof, wherein the composition is formulated for intranasal administration such that such that an amount of rasagiline which is sufficient to inhibit MAO-A in the brain is administered.

According to an aspect of some embodiments of the present invention there is provided a method of treating Parkinson's disease and/or depression associated with Parkinson's disease in a subject in need thereof, the method comprising administering to the subject a pharmaceutical composition comprising Rasagiline or a pharmaceutically acceptable salt thereof, wherein the administering is effected by intranasal administration such that an amount of rasagiline which is sufficient to inhibit MAO-A in the brain is administered.

According to some embodiments of the present invention, the amount of rasagiline or a pharmaceutically acceptable salt thereof is lower than an amount equivalent to 10 mg/kg per day in rats.

According to some embodiments of the present invention, the pharmaceutical composition further comprises a pharmaceutically acceptable carrier, the carrier being a solid carrier or a liquid carrier.

According to an aspect of some embodiments of the present invention there is provided a pharmaceutical composition comprising Rasagiline or a pharmaceutically acceptable salt thereof and a pharmaceutically acceptable carrier, the composition being formulated for intranasal administration.

According to some embodiments of the present invention, the composition is identified for use in the treatment of Parkinson's disease and/or depression associated with Parkinson's disease in a subject in need thereof.

According to some embodiments of the present invention, the composition is identified for use in the treating or motor symptoms and depression symptoms associated with Parkinson's disease.

According to some embodiments of the present invention, the carrier is a solid carrier or a liquid carrier.

According to an aspect of some embodiments of the present invention there is provided a device or system (e.g., delivery system) configured for intranasal administration of a pharmaceutical composition comprising rasagiline or a pharmaceutically acceptable salt thereof to a subject, the device comprising:
a container comprising the composition comprising the Rasagiline or a pharmaceutically acceptable salt thereof; and
means for dispensing a pre-determined dose of the composition from the container and delivering the dose intranasally, the means being configured such that the dose is capable of treating motor and depression symptoms associated with Parkinson's disease.

According to some embodiments of the present invention, the dose is sufficient to inhibit MAO-A in the brain of the subject.

Unless otherwise defined, all technical and/or scientific tern's used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a bar graph showing brain MAO-A inhibition by rasagiline (0.1 mg/kg and 0.3 mg/kg) administered intraperitonealy (IP) and intranasaly (NAS;) to adult male Sprague Dawley rats (*p<0.05 vs. vehicle treated rats, one way ANOVA with Dunnett post test, n=4).

FIG. 2 is a bar graph showing brain MAO-B inhibition by rasagiline (0.1 mg/kg and 0.3 mg/kg) administered IP and NAS to adult male Sprague Dawley rats **p<0.01 vs. vehicle treated rats, one way ANOVA with Dunnett post test, n=4).

FIG. 3 is a bar graph showing liver MAO-A inhibition by rasagiline (0.1 mg/kg and 0.3 mg/kg) administered IP and NAS to adult male Sprague Dawley rats (*p<0.05 vs. vehicle treated rats, one way ANOVA with Dunnett post test, n=4).

FIG. 4 is a bar graph showing liver MAO-B inhibition by rasagiline (0.1 mg/kg and 0.3 mg/kg) were administered IP and NAS to adult male Sprague Dawley rats **p<0.01 vs. vehicle treated rats, one way ANOVA with Dunnett post test, n=4).

FIG. 5 is a bar graph showing striatal MAO-A inhibition by intranasal delivery of rasagiline, given in powder or liquid formulation to acute-treated adult male Sprague Dawley rats. Rats were administered intranasally with rasagiline in dextrose powder formulation (0.6 and 6 mg/kg; 5-ml puff), liquid formulation (0.6 and 6 mg/kg) or respective vehicle (control). Results are represented as mean (% of control) ±SEM (n=9-10 animals per group). ***p<0.001 powder vs. liquid delivery.

FIG. 6 is a bar graph showing a dose-depending effect of intranasal delivery of rasagiline, given in powder formulation, on MAO-A activity in striatum and hippocampus in acute-treated adult male Sprague Dawley rats. Rats were administered intranasally with either vehicle or rasagiline in dextrose powder formulation (0.24, 0.6, 1.5 and 6 mg/kg; 5-ml puff). Results are represented as mean (% of control) ±SEM (n=9-10 animals per group). ***p<0.05 vs. controls.

FIG. 7 is a bar graph showing the effect of intranasal administration of rasagiline, given in liquid or powder formulations, on the ratio of striatal/small intestinal MAO-A inhibition in acute-treated adult male Sprague Dawley rats. Rats were intranasally administered either with rasagiline in dextrose powder formulation (0.24, 0.6, 1.5 and 6 mg/kg; 5-ml puff), liquid formulation (0.6 and 6 mg/kg), or respective vehicle (control). Results are represented as the ratio of % of MAO-A inhibition in rat striatum/small intestine; mean ±SEM (n=9-10 animals per group). *p<0.05 powder vs. liquid intranasal delivery.

FIGS. 8A-B are bar graphs showing the effect of intranasal and oral administration of Rasagiline on MAO-A activity in striatum (FIG. 8A) and hippocampus (FIG. 8B) in acute-treated adult male Sprague Dawley rats. Rats were administered either intranasally (IN, dextrose powder formulation, 5-ml puff) or per-os (P.O) with rasagiline or respective vehicle (control). Results are represented as % of MAO-A inhibition of the respective control groups, mean ±SEM (n=9-10 animals per group). ***p<0.001, *p<0.05 I.N vs. P.O delivery.

FIG. 9 is a bar graph showing the effect of intranasal and oral administration of Rasagiline on the ratio of striatal/ small intestinal MAO-A inhibition in acute-treated adult male Sprague Dawley rats. Rats were administered either intranasally (I.N, dextrose powder formulation, 5-ml puff) or per-os (P.O) with rasagiline, or respective vehicle (control). Results are represented as the ratio of % of MAO-A inhibition in rat striatum/small intestine; mean ±SEM (n=9-10 animals per group). **p<0.01, *p<0.05 I.N vs. P.O delivery.

### DESCRIPTION OF SPECIFIC EMBODIMENTS OF THE INVENTION

The present invention, in some embodiments thereof, relates to therapy and, more particularly, but not exclusively, to compositions, methods and devices useful for the treatment of depression and/or motor and depression symptoms of Parkinson's disease.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Parkinson's disease is the second most common neurodegenerative disorder, affecting 1-3 percent of people older than 50 years. Parkinson's disease is characterized by motor symptoms, such as bradykinesia, rigidity, tremor at rest and postural instability, which are associated with degeneration of the nigrostriatal dopaminergic projection.

Depression is a common and potentially debilitating aspect of Parkinson's disease, affecting 50-70 percent of Parkinsonian patients. Depression in Parkinson's disease is demonstrably different from ordinary major depression in terms of gender ratio, age, symptom profile, comorbidity, and chronicity. Treatment of depression in Parkinson's disease entails special concerns related to side effects and drug-drug interactions.

The currently most common drugs for the treatment of Parkinson's disease are the irreversible selective MAO-B inhibitors (e.g., Rasagiline and Selegiline). These drugs are assumed to exert their primary effect in Parkinson's disease (PD) by MAO-B inhibition which results in a slower metabolism of endogenous and exogenous dopamine (DA), thus providing symptomatic benefits (Finberg et al. 1996, 1998, supra).

Irreversible nonselective MAO-AB and selective MAO-A inhibitors are known as anti-depressants. Such drugs can potentiate the cardiovascular effect of the sympathomimetic amine, tyramine, present in many foods. Since tyramine is metabolized by MAO, the inhibition of MAO-A results in uptake of tyramine from circulatory system, which results in hypertensive crisis, known as the “cheese effect”, as a consequence of noradrenaline release from peripheral adrenergic neurons by tyramine.

Rasagiline and Selegiline, which are currently used in the treatment of Parkinson's disease at doses in which MAO-B inhibition is exerted, may also exhibit MAO-A inhibition, however, at doses which are at least 3-fold higher that those required for exhibiting MAO-B inhibition.

Rasagiline, when given orally or IP, does not cause a “cheese effect” at its selective MAO-B inhibitory activity dosage. However, at higher dosage it loses its selectivity and consequently further inhibits MAO-A, thus causing a “cheese effect”. Rasagiline is also contraindicated with several families of anti-depressants, including for example, the SSRIs. Thus, while treatment of Parkinsonian patients with Rasagiline results in alleviation of motor symptoms associated with the dopaminergic system, such a treatment limits the possibilities of alleviating depression symptoms associated with Parkinson's disease.

In a search for improving the current methodologies of treating Parkinson's disease, the present inventors have conceived administering a MAO-B inhibitor such as Rasagiline directly into the brain, at a dose that would affect depression in a subject in need thereof, and hence would affect both motor and depression symptoms associated with Parkinson's disease. The present invention have envisioned administering a MAO-B inhibitor such as rasagiline at a dose which would inhibit both MAO-A and MAO-B in the brain, yet would not inhibit systemic (peripheral) MAO-A. Such a methodology provides for an efficient treatment of depression and particularly, for an efficient treatment of both motor symptoms and depression symptoms associated with Parkinson's disease, presumably due to MAO-A and MAO-B inhibition in the brain, and avoids inhibition of MAO-A in the periphery and the consequent adverse “cheese reaction”.

According to an aspect of some embodiments of the present invention, there is provided a method of treatment, which is effected by administering to the brain of subject a therapeutically effective amount of a MAO-B inhibitor.

According to some embodiments of the present invention, the method is effected by administering to the brain of a subject a MAO-B inhibitor is an amount that is capable of treating depression in a subject in need thereof.

According to some embodiments, a method as described herein is effected by administering to the brain of a subject a MAO-B inhibitor in an amount that is capable of treating (or alleviating) both motor and depression symptoms associated with Parkinson's disease.

According to some embodiments, the amount of the MAO-B inhibitor is sufficient to inhibit MAO-A in the brain of the subject.

Since, as described hereinabove, irreversible MAO-B inhibitors exhibit MAO-A inhibition at doses higher than the doses at which they exhibit MAO-B inhibition, administering to the brain of a subject a MAO-B inhibitor is an amount that is sufficient to inhibit MAO-A in the brain of the subject results in inhibition of both, MAO-A and MAO-B in the brain.

A method as described herein can be used for treating depression, e.g., by means of inhibiting MAO-A in the brain.

A method as described herein can particularly be used for treating Parkinson's disease, and, in some embodiments, for treating motor symptoms and depression symptoms associated with Parkinson's disease in a subject in need thereof.

According to some embodiments, a “subject in need thereof” is a subject suffering from depression.

According to some embodiments, the subject suffers from Parkinson's disease. Such subjects are also referred to herein and in the art as “Parkinsonian” subjects or patients.

According to some embodiments, the subject is a Parkinsonian subject who suffers, in addition to motor symptoms associated with Parkinson's disease, from depression symptoms associated with Parkinson's disease.

As used herein, the term “depression” is interchangeable to the term “depressive illness” and encompasses psychiatric (or mental) conditions known as major depressive disorder, major depression, clinical depression, or simply depression. Such conditions are characterized, for example, by episodes of all-encompassing low mood accompanied by low self-esteem and loss of interest or pleasure in normally enjoyable activities. Depression, or depressive illness, is characterized, for example, by the presence of some or all of following symptoms: (i) depressed mood most of the day, nearly every day, as indicated by either subjective report or observation made by others; (ii) markedly diminished interest or pleasure in all, or almost all, activities most of the day, nearly every day; (iii) significant weight loss when not dieting or weight gain, or decrease or increase in appetite nearly every day; (iv) insomnia or hypersomnia nearly every day; (v) psychomotor agitation or retardation nearly every day; (vi) fatigue or loss of energy nearly every day; (vii) feelings of worthlessness or excessive or inappropriate guilt nearly every day; (viii) diminished ability to think or concentrate, or indecisiveness, nearly every day; and (ix) recurrent thoughts of death, recurrent suicidal ideation without a specific plan, or a suicide attempt or a specific plan for committing suicide.

In some embodiments, depression can be determined either behaviorally, according to the above-indicated symptoms, or by well-known tests, such as, but not limited to, according to DSM-IV diagnostic criteria.

Hereinthroughout, the term “depression” or “depressive illness” refers to both depression as a psychiatric condition per se, and depression symptoms associated with Parkinson's disease.

As used herein, motor symptoms associated With Parkinson's disease include, for example, bradykinesia, rigidity, tremor at rest and postural instability.

According to an aspect of some embodiments of the present invention, there is provided a method of treating Parkinson's disease in a subject in need thereof, the method comprising administering to the brain of the subject a pharmaceutical composition comprising a MAO-B inhibitor.

According to an aspect of some embodiments of the present invention there is provided a method of treating motor symptoms and depression symptoms associated with Parkinson's disease in a subject in need thereof, the method comprising administering to the brain of the subject a pharmaceutical composition comprising a MAO-B inhibitor.

According to an aspect of some embodiments of the present invention, there is provided a method of treating depression in a subject in need thereof, the method comprising administering to the brain of the subject a pharmaceutical composition comprising a MAO-B inhibitor.

In some embodiments, the depression is associated with Parkinson's disease and the subject is a Parkinsonian subject, as defined herein.

According to an aspect of some embodiments of the present invention, there is provided a use of a MAO-B inhibitor in the manufacture of a pharmaceutical composition (a medicament) for the treatment of Parkinson's disease in a subject in need thereof, the pharmaceutical composition being formulated for administration into the brain of the subject.

According to an aspect of some embodiments of the present invention, there is provided a use of a MAO-B inhibitor in the manufacture of a pharmaceutical composition (a medicament) for the treatment of motor symptoms and depression symptoms associated with Parkinson's disease in a subject in need thereof, the pharmaceutical composition being formulated for administration into the brain of the subject.

According to an aspect of some embodiments of the present invention, there is provided a use of a MAO-B inhibitor in the manufacture of a pharmaceutical composition (a medicament) for the treatment of depression in a subject in need thereof, the pharmaceutical composition being formulated for administration into the brain of the subject.

In some embodiments, the depression is associated with Parkinson's disease and the subject is a Parkinsonian subject, as defined herein.

According to an aspect of some embodiments of the present invention, there is provided a pharmaceutical composition comprising a MAO-B inhibitor, for use in the treatment of Parkinson's disease in a subject in need thereof, the pharmaceutical composition being formulated for administration into the brain of the subject.

According to an aspect of some embodiments of the present invention, there is provided a pharmaceutical composition comprising use of a MAO-B inhibitor, for use in the treatment of motor symptoms and depression symptoms associated with Parkinson's disease in a subject in need thereof, the pharmaceutical composition being formulated for administration into the brain of the subject.

According to an aspect of some embodiments of the present invention, there is provided a pharmaceutical composition comprising a MAO-B inhibitor, for use in the treatment of depression in a subject in need thereof, the pharmaceutical composition being formulated for administration into the brain of the subject.

In some embodiments, the depression is associated with Parkinson's disease and the subject is a Parkinsonian subject, as defined herein.

In some of any of the embodiments of the methods, uses and compositions described herein, the MAO-B inhibitor is used in an amount that is capable of treating depression and/or depressive illness symptoms associated with Parkinson's disease.

In some of any of the embodiments of the methods, uses and compositions described herein, the MAO-B inhibitor is used in an amount that is sufficient to inhibit MAO-A inhibition in the brain of the subject.

Thus, according to some embodiments of any one of the methods as described herein, the MAO-B inhibitor is administered in a therapeutically effective amount, and the therapeutically effective amount is as an amount sufficient to treat or alleviate depression and/or depressive illness symptoms associated with Parkinson's disease.

Thus, according to some embodiments of any one of the methods as described herein, the MAO-B inhibitor is administered in a therapeutically effective amount, and the therapeutically effective amount is as an amount sufficient to inhibit MAO-A in the brain of the subject.

According to some embodiments of any of the compositions and uses as described herein, the composition is used such that an amount of the MAO-B inhibitor administered to the brain of the subject in a therapeutically effective amount as described herein.

According to some embodiments of any of the compositions and uses as described herein, the composition is used such that an amount of the MAO-B inhibitor sufficient to inhibit MAO-A in the brain of the subject.

According to some embodiments of any of the compositions and uses as described herein, the composition comprises a therapeutically effective amount of the MAO-B inhibitor, as described herein.

According to some embodiments of any of the compositions and uses as described herein, the composition comprises an amount of the MAO-B inhibitor is sufficient to inhibit MAO-A in the brain of the subject.

By “therapeutically effective amount” it is generally meant herein an amount effective to treat, alleviate or ameliorate a disorder or a symptom, or prolong the survival of the subject being treated. In the context of embodiments of the present invention, which relate to an amount effective to treat depression and/or Parkinson's disease and/or symptoms associated with Parkinson's disease, the phrase “therapeutically effective amount” describes an amount of a MAO-B inhibitor which is sufficient to alleviate depression and/or depression symptoms associated with Parkinson's disease.

By “amount sufficient to inhibit MAO-A in the brain of the subject” it is meant that a pharmaceutical composition comprising a MAO-B inhibitor as described herein comprises an amount of the MAO-B inhibitor which, when administered, inhibits MAO-A in the brain.

The pharmaceutical composition is used at doses and regimens which provide a therapeutically effective amount of the MAO-B inhibitor in the brain of the subject, and in some embodiments, such a therapeutically effective amount causes inhibition of MAO-A in the brain, as is described in further detail hereinunder.

In some embodiments of any of the embodiments described herein for any one of the methods, compositions and uses described herein, the therapeutically effective amount of the MAO-B inhibitor is such that inhibits MAO-A in the brain yet, and administering the composition does not cause “cheese reaction”, as described herein. In some embodiments, administering the composition does not result in inhibition of systemic MAO-A (MAO-A in the periphery) or results in reduced inhibition of systemic MAO-A.

In some embodiments of any of the embodiments described herein for any one of the methods, compositions and uses described herein, the composition is used such that inhibition of MAO-A in the brain is effected yet, “cheese reaction” is not caused. In some embodiments, administering the composition does not result in inhibition of systemic MAO-A (MAO-A in the periphery) or results in reduced inhibition of systemic MAO-A.

The administration of MAO-B inhibitor into the brain, thus bypassing the liver and small intestine, preferably by intranasal administration, allows using wide range of dosing, wherein high doses of the drug will inhibit MAO-A (and MAO-B) in the brain without potentiation of sympathetic cardiovascular activity, i.e., without the side effect resulting from MAO-A inhibition and produced by said MAO-B inhibitor when administered at high doses to the periphery and ingested together with a high tyramine content food.

In some of any of the embodiments described herein, the pharmaceutical composition as described herein inhibits both MAO-B and MAO-A in the brain.

In some of any of the embodiments described herein, the concentration of the pharmaceutically active agent, i.e., the MAO-B inhibitor as defined herein, in a pharmaceutical composition is determined in accordance with the particular agent chosen; its efficacy; a comparison of its bioavailability by the particular mode of administration used, e.g., intranasal administration, and by other routes of administration, e.g., parenteral injection or oral administration; and the desired frequency of administration combined with the desired single dosage of the formulation. Such pharmacological data can routinely be obtained by the skilled artisan from animal experiments, e.g., in terms of index values. Exemplary animal experiments are provided in the Examples section that follows.

The dosage administered, for example, to a particular Parkinsonian patient will depend on the state of that patient, and will be determined as deemed appropriate by the practitioner.

Thus, according to some of any of the embodiments of the present invention, the pharmaceutical composition is formulated and used (administered to the brain at a certain regimen) so as to deliver to the brain a MAO-B inhibitor as described herein in an amount that inhibits MAO-A in the brain. As discussed herein, such an amount also inhibits MAO-B in the brain.

In some of these embodiments, the pharmaceutical composition is formulated and used (administered to the brain at a certain regimen) so as to deliver to the brain a MAO-B inhibitor as described herein in an amount that does not substantially inhibit systemic MAO-A, namely, MAO-A present in the periphery, for example, in liver and small intestine.

In any one of the compositions, uses and methods as described herein, the MAO-B inhibitor used (or a composition comprising the MAO-B inhibitor) is administered directly into the brain.

Without being bound by any particular theory, administration of MAO-B inhibitor or the brain bypasses the circulation and in particular the liver and small intestine thus avoiding the undesired side effect produced by inhibition of the MAO-A enzyme in these tissues.

In some of any of the embodiments described herein, administration (delivery) of the MAO-B inhibitor is carried out by intrastriatal administration, i.e., directly to the corpus striatum of the individual treated.

In a preferred embodiment, administration (delivery) of the MAO-B inhibitor is carried out by intranasal (also known as nasal) administration. In these embodiments, a composition as described herein is formulated for intranasal administration.

Intranasal administration is a noninvasive means for targeting the brain by passing the BBB, minimizing systemic absorption, and limiting potential peripheral side effects. Intranasal administration allows the drug administered to travel through the roof of the nose, along the fibers of the olfactory and trigeminal nerves found in the mucosa of the nose, directly to the extracellular space of the neurons of the brain and spinal cord without having to cross the BBB or access the blood stream, and consequently, without exposing the other organs of the body to the drug, thus reducing its side effects and required dosage.

Administration of the composition as described herein can be effected once daily, or twice daily, or once every two days. In some embodiments, administration is effected so as to maintain an amount of the MAO-B inhibitor in the brain of the subject, which inhibits MAO-A in the brain, as described herein.

As used herein, a “pharmaceutical composition” refers to a preparation of an active compound (e.g., a MAO-B inhibitor), with other chemical components such as pharmaceutically acceptable and suitable carriers and excipients. The purpose of a pharmaceutical composition is to facilitate administration of a compound to an organism, i.e., to the brain of the subject, as described herein.

Hereinafter, the term “pharmaceutically acceptable carrier” refers to a carrier or a diluent that does not cause significant irritation to an organism and does not abrogate the biological activity and properties of the administered compound. Examples, without limitations, of carriers are: propylene glycol, saline, emulsions and mixtures of organic solvents with water, as well as solid (e.g., powdered) and gaseous carriers.

Herein the term “excipient” refers to an inert substance added to a pharmaceutical composition to further facilitate administration of a compound. Examples, without limitation, of excipients include calcium carbonate, calcium phosphate, various sugars (e.g., dextrose) and types of starch, cellulose derivatives, gelatin, vegetable oils and polyethylene glycols.

In some embodiments, the pharmaceutical composition is identified for administration once per day (e.g., as described herein).

In some embodiments, the pharmaceutical composition comprises a unit dosage form, comprising a therapeutically effective amount of a MAO-B inhibitor, as described herein.

The term “unit dosage form”, as used herein, describes physically discrete units, each unit containing a predetermined quantity of MAO-B inhibitor calculated to produce the desired therapeutic effect, in association with at least one pharmaceutically acceptable carrier, diluent, excipient, or combination thereof.

In some embodiments, the amount of MAO-B inhibitor in the unit dosage form may optionally be a daily dosage of the MAO-B inhibitor, as described herein, such that a method or treatment such as described herein may be effected by administration of one unit dosage form per day.

Alternatively, the amount of the MAO-B inhibitor in the unit dosage form may be, for example, half a daily dosage described herein, such that a method or treatment described herein may be effected by administration of two unit dosage forms per day; or a third or a quarter of a daily dosage described herein, such that a method or treatment described herein may be effected by administration of three or four unit dosage forms per day, respectively.

Further alternatively, the pharmaceutical composition is formulated such that a single dosage of the composition contains a desired amount of the MAO-B inhibitor, as described herein, or can be formulated into a device or a delivery system that dispenses or releases a desired amount of the MAO-B inhibitor as described herein into the brain of a subject.

The pharmaceutical composition comprising the MAO-B inhibitor according to any one of the embodiments relating to the methods, uses and compositions, as described herein, can be prepared by conventional techniques, e.g., as described in Remington: The Science and Practice of Pharmacy, 19th Ed., 1995.

Pharmaceutical compositions of the present invention may be manufactured by processes well known in the art, e.g., by means of conventional mixing, dissolving, granulating, levigating, emulsifying, encapsulating, entrapping or lyophilizing processes.

Pharmaceutical compositions for use in accordance with the present invention thus may be formulated in conventional manner using one or more pharmaceutically acceptable carriers comprising excipients and auxiliaries, which facilitate processing of the MAO-B inhibitor into preparations which can be used pharmaceutically and administered to the brain of a subject. Proper formulation is dependent upon the route of administration chosen.

The compositions can be prepared, e.g., by uniformly and intimately bringing the active agent, i.e., a MAO-B inhibitor, as defined above, into association with a pharmaceutically acceptable carrier, such as a liquid carrier, a (e.g., finely divided) solid carrier, or both, and then, if necessary, shaping the product into the desired formulation.

Depending on the carrier selected, the composition may be in liquid, solid or semisolid form and may further include pharmaceutically acceptable fillers, carriers, diluents or adjuvants, and other inert ingredients and excipients.

In some of any one of the embodiments described herein, the pharmaceutical composition of the present invention is formulated in a solid form, e.g., as a powder and/or as nanoparticles.

The pharmaceutical composition can be formulated for any suitable route of administration that may deliver the active agent directly into the brain, as described herein, and is preferably formulated for intranasal administration.

A pharmaceutical composition formulated for intranasal administration may be liquid, e.g., adapted for administration as a spray or drops. Liquid preparations, such as those based on aqueous formulations, may include ancillary agents, e.g., a pH-buffering system, for example, a buffer such as phosphate, borate, citrate or acetate buffers, a preservative, and an osmotic pressure controlling agent, e.g., glycerol or sodium chloride.

Non limiting examples of buffering agents/systems include boric acid, sodium bicarbonate, sodium citrate, sodium acetate, sodium phosphate monobasic, sodium phosphate dibasic, sodium phosphate dibasic heptahydrate, potassium dihydrogen phosphate, and combinations thereof such as combinations of boric acid and sodium bicarbonate, sodium phosphate monobasic and sodium phosphate dibasic, or sodium citrate and citric acid. If a buffering agent is employed, it is chosen in quantities that preferably do not irritate the nasal mucosa.

In some of these embodiments, the carrier is an aqueous carrier, e.g., water. Such preparations may be prepared by dispersing the active agent, i.e., the MAO-B inhibitor as defined herein, and ancillary agents, utilizing any method usually employed for suspension or emulsification, e.g., ultrasonic treatment. Adjustment of the aqueous phase to neutrality, i.e., to pH in the range from about 6.5 to about 8, may be accomplished in any of the preparatory steps.

In some embodiments, microemulsions for intranasal administration are prepared in which the size of the dispersed particles or droplets is of the order of 10 nm, thereby facilitating their passage across the nasal mucosa. Such microemulsions may be sterilized by filtration.

In certain embodiments, the pharmaceutical composition includes one or more agents that increase viscosity, chosen in quantities that preferably do not irritate the nasal mucosa and increase nasal retention time. Examples of agents that increase viscosity include, without being limited to, methylcellulose, carboxymethylcellulose sodium, ethylcellulose, carrageenan, carbopol, and combinations thereof.

The pharmaceutical composition may contain aqueous diluents, e.g., saline, water, dextrose, and combinations thereof, and/or non-aqueous, e.g., alcohols, particularly polyhydroxy alcohols such as propylene glycol, polyethylene glycol, and glycerol, vegetable oils and mineral oils. These aqueous and non-aqueous diluents can be added in various concentrations and combinations to form solutions, suspensions, oil-in-water emulsions or water-in-oil emulsions.

The pH of the compositions may be adjusted to the desired value using any suitable organic or inorganic acid or organic or inorganic base. Suitable organic acids include, without limiting, acetic acid, citric acid, glutamic acid and methane sulfonic acid. Suitable inorganic acids include, but are not limited to, hydrochloric acid and sulphuric acid. Suitable organic bases include, without limiting, meglumine, lysine and tromethamine. Suitable inorganic bases include, without being limited to, sodium hydroxide and potassium hydroxide.

Solvents that may be used to prepare the pharmaceutical compositions of the invention include, without being limited to, water, ethanol, propylene glycol, polyethylene glycol, glycerin, phenol, glycofurol, benzyl benzoate and polyoxyethylene castor oil derivatives.

In a preferred embodiment of any of the aspects described herein, the pharmaceutical composition is in a solid form, and comprises pharmaceutically acceptable solid carrier.

Suitable carriers and/or excipients include, for example, fillers such as sugars, including dextrose, lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

For administration by inhalation (e.g., intranasal administration), the MAO-B inhibitor can be conveniently delivered in the form of an aerosol spray presentation (which typically includes powdered, liquefied and/or gaseous carrier) from a pressurized pack or a nebulizer, with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in an inhaler or insufflator may be formulated containing a powder mix of MAO-B inhibitor and a suitable powder base such as, but not limited to, lactose or starch.

Pharmaceutically acceptable excipients such as dispersing agents, isotonicity agents, stabilizing agents, and the like can be used as appropriate in the pharmaceutical compositions. The pharmaceutical compositions of the invention may contain excipients such as antioxidants, chemical preservatives, buffering agents, agents that increase viscosity, diluents, pH adjusters, and solvents.

Antioxidants are substances that prevent oxidation of the formulations. Suitable antioxidants for use in the compositions of the invention include, without being limited to, butylated hydroxytoluene, butylated hydroxyanisole, potassium metabisulfite, and the like.

In certain embodiments, the pharmaceutical composition of the present emnbodiments contains a preservative chosen in quantities that preserve the composition but do not cause irritation of the nasal mucosa. Examples of suitable preservatives include, without limiting, benzalkonium chloride, methyl, ethyl, propyl- or butylparaben, benzyl alcohol, phenylethyl alcohol, benzethonium, and combinations thereof.

The pharmaceutical compositions of the present embodiments may contain other pharmaceutically acceptable ingredients well known in the art. Such excipients include, without limiting, chelating agents such as edetic acid or a salt thereof, flavors, sweeteners, thickening, adhesive or gelling agents, e.g., celluloses such as methylcellulose, hydroxypropyl methylcellulose, hydroxypropyl cellulose, sodium carboxyl cellulose and microcrystalline cellulose, poloxomers, polyethylene glycols, carbomers or polyethylene oxide.

The pharmaceutical compositions of the present invention, when formulated for intranasal administration, may be used in any dosage dispensing device adapted for intranasal administration. The device should be constructed with a view to ascertaining optimum metering accuracy and compatibility of its constructive elements.

According to an aspect of some embodiments of the present invention, there is provided a device, or a delivery system, configured for intranasal administration of a pharmaceutical composition comprising a MAO-B inhibitor as described herein to a subject.

In some embodiments, the device is configured for dispensing, from a container comprising the composition, and may comprise means for dispensing a pre-determined dose of the composition from the container and delivering said dose intranasally.

In some embodiments, the pre-determined dose is such that when administered intranasally, results in an amount of the MAO-B inhibitor in the brain which is a therapeutically effective amount, as described herein, and/or is sufficient to inhibit MAO-A in the brain, as described herein.

The compositions may be administered as drops, sprays, aerosols or by any other intranasal dosage form. Optionally, the delivery system may be a unit dose delivery system. The volume of solution, powder or suspension delivered per dose may be anywhere from 10 to 10000 μl and preferably 1000-5000 μl.

Delivery systems for these various dosage forms may be dropper bottles, plastic squeeze units, atomizers, nebulizers, metered nasal sprayers, or pharmaceutical aerosols in either unit dose or multiple dose packages.

Aerosol systems require a propellant to be inert towards the formulation. Suitable propellants may be selected among such gases as fluorocarbons, hydrocarbons, nitrogen and dinitrogen oxide or mixtures thereof.

In some embodiments, the device is configured for dispensing a composition, as described herein, wherein the composition is a solid composition.

According to any one of the embodiments described herein, the MAO-B inhibitor may be any selective irreversible MAO-B inhibitor such as, without being limited to, rasagiline (N-propargyl-1-(R)-aminoindan, Azilect®), used as a monotherapy in early Parkinson's disease or as an adjunct therapy in more advanced cases; selegiline ((R)-N-methyl-N-(1-phenylpropan-2-yl)prop-1-yn-3-amine; L-depreny; Eldepryl), used for the treatment of early-stage Parkinson's disease, depression and senile dementia; safinamide (N2-{4-[(3-fluorobenzyl)oxy]benzyl}-L-alaninamide), or a pharmaceutically acceptable salt thereof.

In particular embodiments, in any of the methods, compositions, uses and devices described herein, the MAO-B inhibitor is rasagiline or a pharmaceutically acceptable salt thereof.

Non-limiting examples of pharmaceutically acceptable salts of Rasagiline include the mesylate salt; the esylate salt; the maleate salt; the fumarate salt; the tartrate salt; the sulfate salt; the hydrochloride salt; the hydro bromide salt; the p-toluenesulfonate salt; the benzoate salt; the acetate salt; or the phosphate salt of rasagiline.

Pharmaceutically acceptable salts of rasagiline may be prepared according to any suitable technique known in the art, e.g., as described in detail in U.S. Pat. No. 5,532,415.

In preferred embodiments, the Rasagiline is used as a mesylate salt of Rasagiline.

In any one of the embodiments described herein, the MAO-B inhibitor (e.g., Rasagiline) can be in any of the possible stereoisomers or enentiomers, or as a mixture of two or more stereoisomer sot enetiomers, or as a racemic mixture.

In some of any the embodiments described herein, an amount of Rasagiline or a salt thereof administered to the brain of the subject is lower than an amount equivalent to 10 mk/kg per day in rats, that is, lower than the amount required for inhibiting MAO-A in the brain when Rasagiline or a salt thereof is administered orally or IP.

In some of any the embodiments described herein, an amount of Rasagiline or a salt thereof administered to the brain of the subject is higher than an amount equivalent to 1 mg per day in humans.

An amount of 1 mg in humans is equivalent to about 0.1 mg/kg in rats.

In some embodiments, an amount of Rasagiline or a salt thereof administered to the brain of the subject is higher than an amount equivalent to 0.1 mg/kg in rats.

In any one of the compositions, methods, uses and devices as described herein, the MAO-B inhibitor can be used in combination with an additional active agent or drug, for example, an anti-Parkinsonian drug such as 1-DOPA.

It is expected that during the life of a patent maturing from this application many relevant MAO-B inhibitors will be developed and the scope of the term MAO-B is intended to include all such new technologies a priori.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

### Example 1

Materials and Methods:

Rasagiline (mesylate salt), was used.

Adult (3-month old) male Sprague Dawley rats, weighing about 250 grams, were from Harlan Laboratories, Inc Israel.

Rasagiline (0.1 and 0.3 mg/kg) and vehicle were administered intraperitonealy (IP) and intranasaly (NAS) to adult male Sprague Dawley rats (n=4 per each experimental group). The animals were sacrificed 1 hour after the treatment; the brains and livers were rapidly removed and dissected; and the effect of rasagiline, given at the two different regimens (IP and NAS) on MAO-A and MAO-B activities was examined in the brain and liver, as previously described (Tipton et al., 1982).

Results:

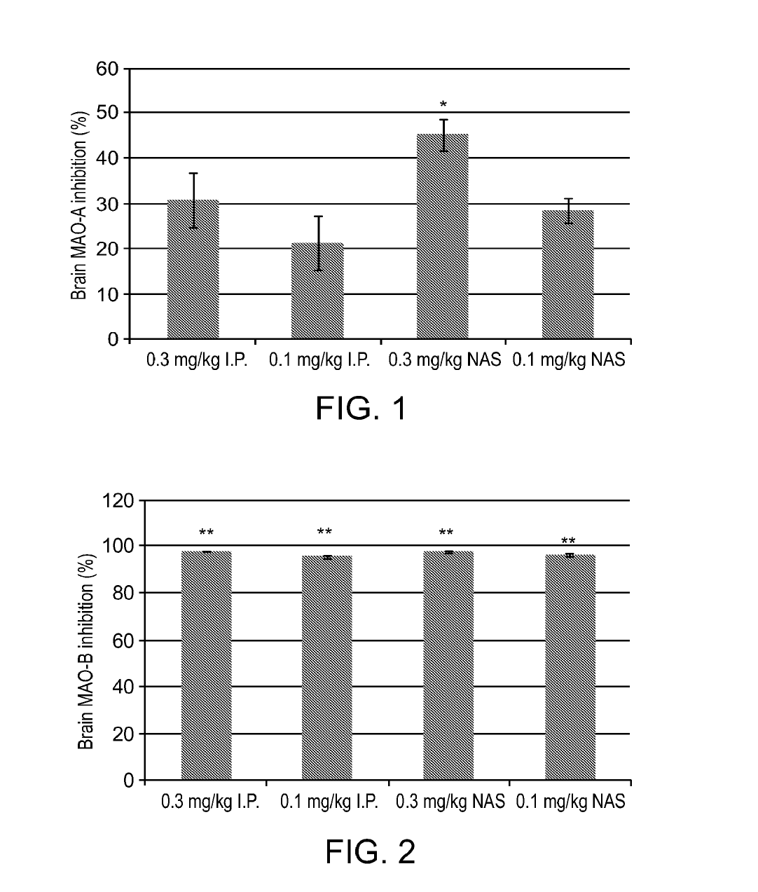

The inhibition of MAO-A and MAO-B in the brain by IP and NAS administration of Rasagiline is presented in FIGS. 1 and 2, respectively.

FIG. 1 demonstrates that rasagiline (0.3 mg/kg) significantly inhibited brain MAO-A inhibition by using NAS delivery compared to IP administration. FIG. 2 demonstrates that no significant difference in brain MAO-B inhibition is observed between the two modes of drug administration.

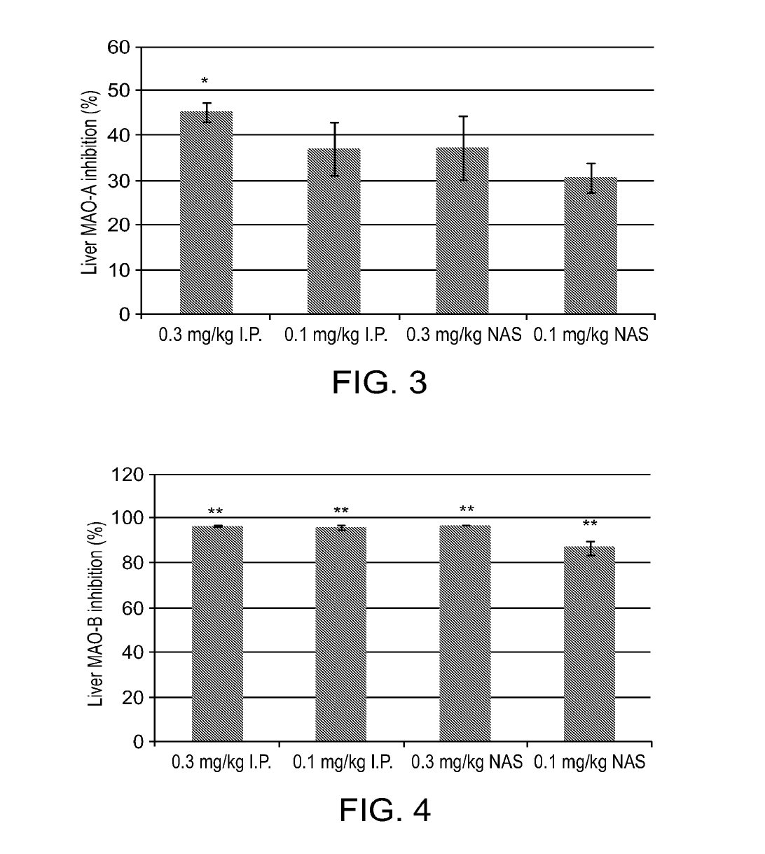

The inhibition of MAO-A and MAO-B in the liver by IP and NAS administration of Rasagiline is presented in FIGS. 3 and 4, respectively.

FIG. 3 demonstrates that rasagiline (0.3 mg/kg) significantly inhibited liver MAO-A inhibition when administered IP, compared to NAS delivery. No significant differences in liver MAO-B inhibition were observed between the two modes of drug administration, as shown in FIG. 4.

### Example 2

Intranasal administration of powder and liquid formulations of Rasagiline (as a mesylate salt) was tested for inhibition MAO-A in the brain and periphery.

Materials and Methods:

All procedures were carried out in accordance with the National Institutes of Health Guide for care and Use of Laboratory Animals, and were approved by the Animal Ethics Committee of the Technion, Haifa, Israel.

A mesylate salt of Rasagiline was used in all experiments. Powder formulation of rasagiline was prepared using a dextrose filler. Liquid formulation of rasagiline was prepared in water as a vehicle. Control liquid formulations included vehicle only.

Adult (3 months-old; about 250 grams weight) male Sprague Dawley rats (obtained from Harlan Laboratories, Inc Israel) were administered intranas ally with a 5 ml puff of a powder formulation of rasagiline in dextrose (0.24, 0.6, 1.5 and 6 mg/kg) or liquid formulation of rasagilone in water vehicle (0.6 and 6 mg/kg), or respective vehicle (controls). The animals were acutely administered intranasally and sacrificed 4 hours following drug/vehicle administration. The brain regions, hippocampus and striatum, as well as the small intestine were dissected out. All tissues were frozen at −80° C. for further analysis.
The effect of different doses of intranasal treatment of rasagiline (in powder formulation and in liquid formulation) on MAO-A and MAO-B activity, was measured in the striatum, hippocampus and small intestine, according to Tipton et. al. (1982). In brief, samples were dissected out at 4° C. and homogenized. Homogenates were incubated with [C¹⁴]serotonin for 30 minutes (final concentration 100 μM) as a substrate for MAO-A, or with [C¹⁴]phenylethylamine for 20 minutes (final concentration 100 μM) as a substrate for MAO-B. The radioactivity was determined by liquid- scintillation.

Results:

The effect of acute intranasal delivery of rasagiline, given in powder or liquid formulation, on rat striatal and hippocampal MAO-A and MAO-B and on striatum/small intestine MAO-A ratio, was measured.

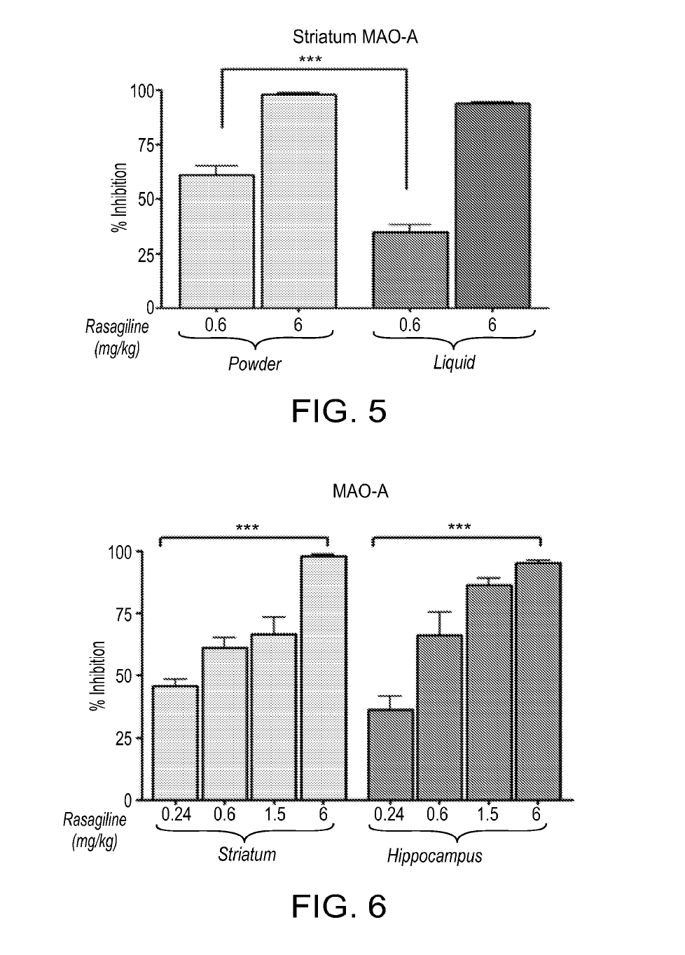

The obtained data is presented in FIGS. 5-7 and in Tables 1-3.

As shown in FIG. 5 and Table 1, intranasal (I.N.) delivery of rasagiline in powder formulation at 0.6 mg/kg caused a significant higher inhibition of striatal MAO-A activity, compared to the same dose of rasagiline delivered in liquid formulation in rats.

TABLE 1
(MAO-A inhibition)

| Treatment | | | | | |
|---|---|---|---|---|---|
| (dose and | | | | | |
| formulation) | Brain | Striatum | Hippocampus | Intestine | Liver |
| 0.6 mg/kg Powder | 44% | 61% | 66% | 42% | 44% |
| 1.5 mg/kg Powder | n.d. | 67% | 86% | 74% | 77% |
| 6 mg/kg Powder | 96% | 98% | 93% | 92% | 94% |
| 0.6 mg/kg Liquid | 33% | 35% | n.d. | 32% | 19% |
| 6 mg/kg Liquid | 91% | 94% | n.d. | 86% | 83% |

As further shown in Table 1, and in FIG. 6, intranasal (I.N.) treatments of rasagiline (0.24, 0.6, 1.5 and 6 mg/kg) in powder formulation dose-dependently inhibited MAO-A activity in rat striatum and hippocampus, while more than 90% MAO-A inhibition was observed at a dose of 6 mg/kg (in powder formulation).

Table 2 below presents the data obtained for MAO-B inhibition. As shown therein, Rasagiline (at doses ranging from 0.24 to 6 mg/kg) at both powder and liquid formulations, significantly inhibited (about 98%; p<0.05) MAO-B activity in the striatum and hippocampus of drug-treated rats, compared to respective vehicle-treated animals (not shown).

TABLE 2
(MAO-B Inhibition)

| Treatment | | | | | |
|---|---|---|---|---|---|
| (dose and formulation) | Brain | Striatum | Hippocampus | Intestine | Liver |
| 0.6 mg/kg Powder | 96% | | 97% | 68% | 95% |
| 1.5 mg/kg Powder | n.d. | 98% | 98% | 72% | 98% |
| 6 mg/kg Powder | 98% | | 99% | 78% | 98% |
| 0.6 mg/kg Liquid | 96% | | n.d. | 63% | 93% |
| 6 mg/kg Liquid | 99% | | n.d. | 75% | 98% |

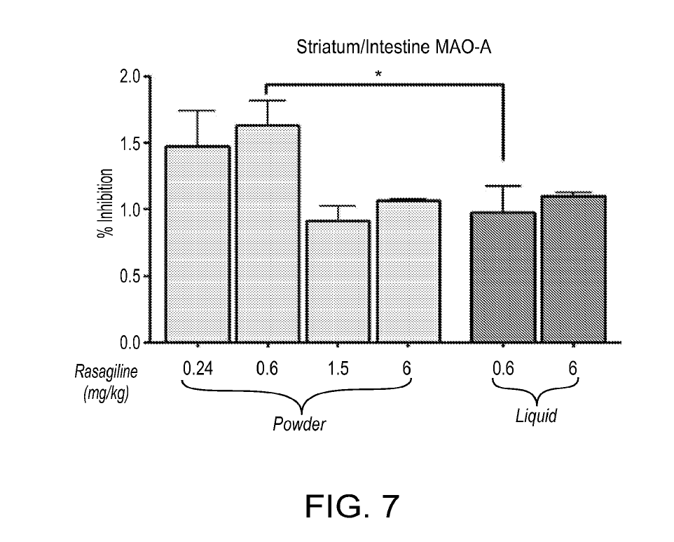

Table 3 below, and FIG. 7, present the ratio of MAO-A inhibition in the CNS vs. the periphery, represented by the ratio of MAO-A inhibition in striatum/Hippocampus and intestines or striatum/Hippocampus and liver, upon intranasal (I.N.) delivery of rasagiline, at variables doses in powder formulation. As shown therein, a dose of 0.6 mg/kg intranasally delivered Rasagiline resulted in the highest ratio of % MAO-A inhibition in the striatum vs. small intestine.

TABLE 3
(Ratio of CNS/Peripheral MAO-A inhibition)

| Treatment | | | Hippo- | |
|---|---|---|---|---|
| (dose and | Striatum/ | Striatum/ | campus/ | Hippocampus/ |
| formulation) | Intestine | Liver | Intestine | Liver |
| 0.6 mg/kg Powder | 1.63 | 1.47 | 1.87 | 1.65 |
| 1.5 mg/kg Powder | 0.911 | 0.87 | 1.17 | 1.12 |
| 6 mg/kg Powder | 1.06 | 1.04 | 1.03 | 1.00 |
| 0.6 mg/kg Liquid | 0.98 | 1.06 | n.d. | n.d. |
| 6 mg/kg Liquid | 1.10 | 1.13 | n.d. | n.d. |

### Example 3

Studies were conducted for determining the potency of intranasal vs. oral administration of Rasagiline in inhibition of MAO-A in the striatum.

Materials and Methods:

All procedures were carried out in accordance with the National Institutes of Health Guide for care and Use of Laboratory Animals, and were approved by the Animal Ethics Committee of the Technion, Haifa, Israel.

A mesylate salt of Rasagiline was used.

Powder formulation of rasagiline was prepared in dextrode.

Liquid oral foiiiiulation of rasagiline was prepared in water as a vehicle.

Control liquid formulations included vehicle only.

Adult (3 months-old; about 250 grams weight) male Sprague Dawley rats (obtained from Harlan Laboratories, Inc Israel), weighing approx. 250 grams were administered intranasally (IN) a 5 ml puff of Rasagiline (mesylate salt), prepared in dextrose as powder formulation, per-os (P.O) Rasagiline prepared in water) (0.24 and 0.6 mg/kg) or respective vehicle (controls). The animals were acutely administered and sacrificed 4 hours following drug/vehicle administration. The brain regions, hippocampus and striatum, as well as the small intestine were dissected out. All tissues were frozen at −80° C. for further analysis.

The effect of different doses of intranasal or oral treatment of rasagiline (0.24 and 0.6 mg/Kg) on MAO-A and MAO-B activity in acute-treated rats, was measured according to Tipton et. al. (1982, supra).

Results:

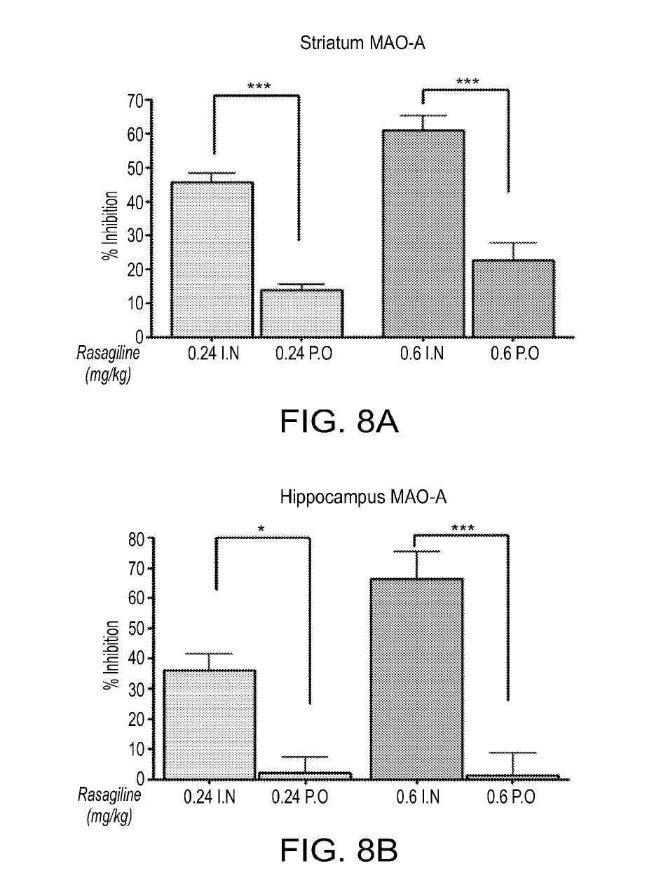

FIGS. 8A and 8B present the effects of acute intranasal delivery vs. oral administration of rasagiline on rat striatal and hippocampal MAO-A and MAO-B, respectively, and the ratio of striatal/small intestinal MAO-A inhibition, respectively.

As shown therein, Rasagiline at both tested concentrations (0.24 and 0.6 mg/Kg) significantly increased MAO-A inhibition activity in rat striatum and hippocampus following intranasal drug delivery (powder formulation), compared to oral administration. Rasagiline at both regimens significantly inhibited (about 99%; p<0.05) MAO-B activity in the striatum and hippocampus of drug-treated rats (data not shown).

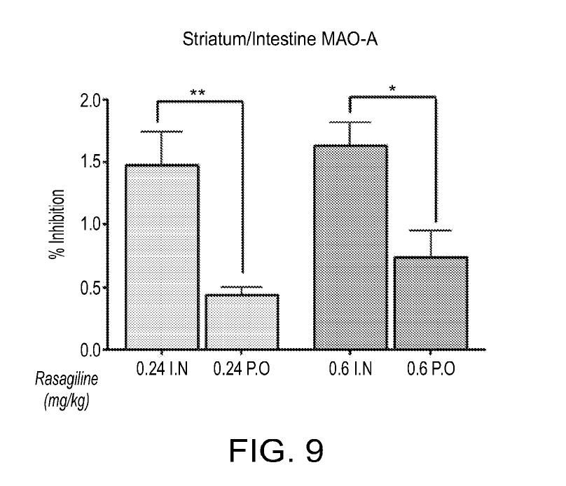

FIG. 9 presents the ratio of % MAO-A inhibition in the striatum/small intestine, and shows that Rasagiline exerted significantly lower MAO-A inhibition in the periphery (small intestine) following intranasal delivery, compared to P.O. administration.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

What is claimed is:

1. A method of treating motor symptoms, non motor symptoms and depression symptoms associated with Parkinson's disease in a subject in need thereof, the method comprising administering to said subject a pharmaceutical composition comprising rasagiline or a pharmaceutically acceptable salt thereof, wherein said administering is effected by intranasal administration, and wherein a daily dose of said rasagiline or pharmaceutically acceptable salt thereof in said pharmaceutical composition is sufficient to inhibit monoamine oxidase (MAO)-A and MAO-B in a brain but insufficient to inhibit systemic MAO-A and therefore does not potentiate sympathetic cardiovascular activity associated with tyramine rich food consuming.

2. The method of claim 1, wherein said pharmaceutically acceptable salt of rasagiline is a mesylate salt, an esylate salt, a maleate salt, a fumarate salt, a tartrate salt, a sulfate salt, a hydro bromide salt, a p-toluenesulfonate salt, a benzoate salt, an acetate salt, or a phosphate salt of rasagiline.

3. The method of claim 1, wherein the daily dose of said rasagiline or pharmaceutically acceptable salt thereof in said pharmaceutical composition is less than 100 mg.

4. The method of claim 3, wherein the daily dose of said rasagiline or pharmaceutically acceptable salt thereof in said pharmaceutical composition is lower than an amount equivalent to about 1.5 mg/kg per day in a rat.

5. The method of claim 4, wherein the daily dose of said rasagiline or pharmaceutically acceptable salt thereof in said pharmaceutical composition is equivalent to about 0.6 mg/kg per day in a rat.

6. The method of claim 1, wherein said pharmaceutical composition is formulated as a powder or liquid.

7. The method of claim 6, wherein said pharmaceutical composition is formulated as nanoparticles.

8. The method of claim 1, wherein said pharmaceutical composition is administered to said subject once daily.

9. The method of claim 1, wherein said pharmaceutical composition further comprises a pharmaceutically acceptable solid or liquid carrier such as a sugar.

10. The method of claim 9, wherein said sugar is dextrose, lactose, sucrose, mannitol, or sorbitol.

11. A method of treating Parkinson's disease and/or depression associated with Parkinson's disease in a subject in need thereof, the method comprising administering to said subject a pharmaceutical composition comprising rasagiline or a pharmaceutically acceptable salt thereof, wherein said administering is effected by intranasal administration, and wherein a daily dose of said rasagiline or pharmaceutically acceptable salt thereof in said pharmaceutical composition is sufficient to inhibit monoamine oxidase (MAO)-A and MAO-B in a brain but insufficient to inhibit systemic MAO-A and therefore does not potentiate sympathetic cardiovascular activity associated with tyramine rich food consuming.

12. The method of claim 11, wherein said pharmaceutically acceptable salt of rasagiline is a mesylate salt, an esylate salt, a maleate salt, a fumarate salt, a tartrate salt, a sulfate salt, a hydro bromide salt, a p-toluenesulfonate salt, a benzoate salt, an acetate salt, or a phosphate salt of rasagiline.

13. The method of claim 11, wherein the daily dose of said rasagiline or pharmaceutically acceptable salt thereof in said pharmaceutical composition is less than 100 mg.

14. The method of claim 13, wherein the daily dose of said rasagiline or pharmaceutically acceptable salt thereof in said pharmaceutical composition is lower than an amount equivalent to about 1.5 mg/kg per day in a rat.

15. The method of claim 14, wherein the daily dose of said rasagiline or pharmaceutically acceptable salt thereof in said pharmaceutical composition is equivalent to about 0.6 mg/kg per day in a rat.

16. The method of claim 11, wherein said pharmaceutical composition is formulated as a powder or liquid.

17. The method of claim 16, wherein said pharmaceutical composition is formulated as nanoparticles.

18. The method of claim 11, wherein said pharmaceutical composition is administered to said subject once daily.

19. The method of claim 11, wherein said pharmaceutical composition further comprises a pharmaceutically acceptable solid or liquid carrier such as a sugar.

20. The method of claim 19, wherein said sugar is dextrose, lactose, sucrose, mannitol, or sorbitol.

21. A pharmaceutical composition comprising rasagiline or a pharmaceutically acceptable salt thereof and a pharmaceutically acceptable carrier, wherein said composition being formulated for intranasal administration, and wherein a dose of said rasagiline or pharmaceutically acceptable salt thereof in said composition is sufficient to inhibit monoamine oxidase (MAO)-A and MAO-B in a brain but insufficient to inhibit systemic MAO-A and therefore does not potentiate sympathetic cardiovascular activity associated with tyramine rich food consuming.

22. The pharmaceutical composition of claim 21, wherein the dose of said rasagiline or pharmaceutically acceptable salt thereof in said composition is less than 100 mg.

23. The pharmaceutical composition of claim 22, wherein the dose of said rasagiline or pharmaceutically acceptable salt thereof in said composition is lower than an amount equivalent to 1.5 mg/kg per day in a rat.

24. The pharmaceutical composition of claim 23, wherein the dose of said rasagiline or pharmaceutically acceptable salt thereof in said composition is equivalent to about 0.6 mg/kg per day in a rat.

25. The pharmaceutical composition of claim 21, wherein said pharmaceutical composition is formulated as a powder or liquid.

26. The pharmaceutical composition of claim 25, wherein said pharmaceutical composition is formulated as nanoparticles.

27. The pharmaceutical composition of claim 21, wherein said pharmaceutical composition further comprises a pharmaceutically acceptable solid or liquid carrier such as a sugar.

28. The pharmaceutical composition of claim 27, wherein said sugar is dextrose, lactose, sucrose, mannitol, or sorbitol.

29. A device configured for intranasal administration of a pharmaceutical composition comprising rasagiline or a pharmaceutically acceptable salt thereof to a subject, the device comprising:
a container comprising the composition comprising said rasagiline or pharmaceutically acceptable salt thereof; and
a dispenser is configured to dispense a pre-determined dose of said composition from said container and delivering said dose intranasally, said dispenser being configured such that said dose is capable of inhibiting monoamine oxidase (MAO)-A and MAO-B in a brain thereby treating motor symptoms, non motor symptoms and depression symptoms associated with Parkinson's disease, but insufficient to inhibit systemic MAO-A and therefore does not potentiate sympathetic cardiovascular activity associated with tyramine rich food consuming.
